(19) United States
(12) Patent Application Publication
(10) Pub. No.: US 2011/0182869 A1
(43) Pub. Date: Jul. 28, 2011

(54) TREATMENT OF GUT MOTILITY DISORDERS

(75) Inventors: Jan Knol, Wageningennl (NL); Kaouther Ben Amor, Utrecht (NL); Richèle Deodata Wind, Nijmegan (NL)

(73) Assignee: N.V. NUTRICIA

(21) Appl. No.: 13/003,990

(22) PCT Filed: Jul. 14, 2009
(86) PCT No.: PCT/NL09/50426

§ 371 Date: Apr. 8, 2011

(30) Foreign Application Priority Data
Jul. 15, 2008 (NL) PCTNL2008050478

(51) Int. Cl. A61K 35/74; A61K 35/20; A61K 31/7008; A61K 31/43; A61K 31/397; A61K 31/496; A61K 31/635; A61P 1/00

(52) U.S. Cl. 424/93.45; 424/93.4; 424/535; 514/62; 514/193; 514/210.09; 514/253.08; 514/158

(57) ABSTRACT

This invention relates to products comprising antibiotics or probiotics and the use thereof for the treatment of gut motility disorders such as irritable bowel syndrome (IBS).

### FIELD OF THE INVENTION

This invention relates to products and the use thereof for the treatment of gut motility disorders such as irritable bowel syndrome (IBS).

### BACKGROUND OF THE INVENTION

Irritable bowel syndrome (IBS) is a common gastrointestinal disorder of unknown aetiology. The microbiota is suggested to play a role in IBS. Based on clinical studies, consensus was reached in the field and the Rome Criteria were developed for classifying the symptoms of IBS. The criteria emphasize the presence of abdominal pain and the link between pain and changes in bowel habit. Irritable bowel syndrome is a common intestinal condition characterized by abdominal pain and cramps; changes in bowel movements (diarrhea, constipation or both); gassiness; bloating; nausea; and other symptoms. There is no cure for IBS. Much about the condition remains unknown or poorly understood; however, dietary changes, drugs, and psychological treatment are often able to eliminate or substantially reduce the symptoms. Approximately 10-15% of the general population has IBS, and it affects females more often than males, for unexplained reasons.

Several possibilities have been described for the treatment of irritable bowel syndrome (IBS).

US 2005/053641 describes the use of fibers for the treatment of IBS.

WO 2005/055934 claims the use of Bacillus coagulans for the treatment of IBS.

EP 1384483 discloses the use of probiotics for treatment of IBS through improvement of gut neuromuscular function.

WO 94/04136 describes the use of polysaccharides with anion binding properties for the treatment of IBS.

WO 2004/089115 discloses synbiotic compositions of pro- and prebiotic compounds and its uses for the treatment of IBS.

Faber in American Journal of Gastroenterology, vol. 95, 2000, p 2533 analysed unbalance in gut flora of 26 IBS patients and reported the following pathogenic organisms in stool: Klebsiella pneumoniae (16/26), Citrobacter freundi (15/26), Pseudomonas aeruginosa (3/26) and Klebsiella oxytoca (3/26) and the yeasts Candida albicans (10/26) and Candida glabrata (4/26).

McFarland et al. World J. Gastroenterol, vol 14, May 2008, pp 2650-2661 describe a meta-analysis of published clinical trials on probiotics for the treatment of IBS.

Although it is recognized in the art that the bacterial flora might be an important causative factor for IBS and thus a good option for treatment of IBS, no indication can be found in the literature which constituent of the flora is responsible for the intestinal motility disorders.

### SUMMARY OF THE INVENTION

The present inventors now surprisingly found that the presence of Pseudomonas aeruginosa in the gastro-intestinal tract is associated with gut motility disorders such as IBS. Because of this finding, the treatment of IBS can be more specific than in the past since the treatment can be aimed at modulating, in particular reducing, the number of Pseudomonas aeruginosa bacteria present in the gut. Modulating, or reducing or decreasing the number of Pseudomonas aeruginosa bacteria is also referred to herein as treatment of Pseudomonas aeruginosa overgrowth.

The inventors surprisingly found a higher prevalence and higher quantities of Pseudomonas aeruginosa in mucosa-associated bacteria of the small intestine and faeces of IBS patients than in healthy subjects using. This finding prompted the present inventors to use specific treatments aimed at decreasing and preferably eradicating P. aeruginosa in the small intestine in order to treat IBS.

A possible method for modulating the number of Pseudomonas aeruginosa, preferably in the small intestine, is the use of specific antibiotics. Preferably said specific antibiotics are aimed at eradicating Pseudomonas aeruginosa.

Another possible method for modulating the number of Pseudomonas aeruginosa, preferably in the small intestine, is the use of probiotic bacteria, in particular for reducing and/or preventing Pseudomonas aeruginosa overgrowth, in particular in the small intestine, and hence for use in the treatment of patients with gut motility disorders including in particular irritable bowel syndrome.

Furthermore the inventors found that the compositions according to the present invention comprising probiotic bacteria can be used for the treatment of symptoms related to irritable bowel syndrome including diarrhea, (gastrointestinal) infections, colic's, abdominal cramps and abdominal pain.

In particular the inventors surprisingly found that specific probiotics selected from Lactobacillus fermentum (NumRes 4 and NumRes 2), Lactobacillus casei (CRL-431), Bifidobacterium animalis (BB-12), Bifidobacterium longum biotype longum (BB-536), Lactobacillus casei (DN-114 001=CNCM I-1518), Bifidobacterium animalis (DN-173 010=CNCM I-2494) or a mixture thereof are preferably used for the treatment of gut motility disorders since these selected probiotics have specific beneficial properties such as improved adherence to intestinal lining cells and have strong anti-Pseudomonas activity.

### DETAILED DESCRIPTION OF THE INVENTION

Antibiotic Compositions

Antibiotics can be used for the modulating the number or amount or level of P. aeruginosa in the gut of a patient, in particular in the small intestine. Thus suitably a composition comprising one or more anti-Pseudomonas antibiotics would be very beneficial for use in the treatment of gut motility disorders, in particular for use in the treatment of IBS. In one embodiment the composition comprising one or more anti-Pseudomonas antibiotics is for use in the treatment of infection with Pseudomonas aeruginosa in a patient with a gut motility disorder, preferably for use in the treatment of infection with Pseudomonas aeruginosa in patients with IBS.

Thus in one embodiment the invention concerns a method for the treatment of a gut motility disorder, and preferably for the treatment of IBS, said method comprising administering a composition comprising one or more anti-Pseudomonas antibiotics to a patient in need thereof. In other words the invention relates to the use of a composition comprising one or more anti-Pseudomonas antibiotics for the preparation of a medicament for the treatment of a gut motility disorder, preferably for the treatment of IBS. The invention can also be worded as a composition comprising one or more anti-Pseudomonas for use in the treatment of a gut motility disorder, preferably for use in the treatment of IBS.

It is advantageous to treat Pseudomonas overgrowth with a combination therapy. Thus in one embodiment the antibiotic composition comprises a combination of an anti-Pseudomonas beta-lactam and an aminoglycoside or fluoroquinolone.

Alternatively a preferred composition comprises an antibiotic selected from the gorup consisting of carbapenem, aminoglycoside, Cotrimox, Ciprofloxacin, Norfloxacin or mixtures thereof.

Probiotic Compositions

In an alternative approach, the present inventors found that probiotic bacteria can bring about a decrease of Pseudomonas in the intestinal mucosa. In a preferred embodiment therefore, the present invention concerns a method for the treatment of a gut motility disorder, and preferably for the treatment of IBS, said method comprising administering a composition comprising one or more probiotic bacteria to a patient need thereof. In other words the invention relates to the use of a composition comprising probiotic bacteria for the preparation of a composition, preferably a nutritional composition, for the treatment of a gut motility disorder, preferably for the treatment of IBS. The invention can also be worded as a composition, preferably a nutritional composition, comprising probiotic bacteria for use in the treatment of a gut motility disorder, preferably for use in the treatment of IBS. In a specific embodiment the present invention concerns the treatment with a probiotic composition of Pseudomonas aeruginosa overgrowth in a patient with a gut motility disorder. Preferably the gut motility disorder is IBS. In one embodiment the present invention concerns the treatment with a probiotic composition of Pseudomonas aeruginosa infection in a patient with a gut motility disorder, preferably for the treatment of Pseudomonas aeruginosa infection in a patient with IBS.

In general small intestinal bacterial overgrowth (SIBO) refers to a condition in which abnormally large numbers of bacteria, at least 100,000 bacteria per ml of intestinal fluid, are present in the small intestine and the types of bacteria in the small intestine resemble more the bacteria of the colon than the small intestine.

Overgrowth by Pseudomonas aeruginosa can be established by the method as disclosed in example 1 herein. P. aeruginosa overgrowth is defined as that the bacterial load of P. aeruginosa in the small intestine is higher than the average bacterial load of P. aeruginosa in healthy subjects, in particular healthy subjects who are not diagnosed as having IBS. Preferably Pseudomonas aeruginosa overgrowth is defined as a bacterial load of P. aeruginosa of at least 2% in the small intestine of a subject, preferably a bacterial load of P. aeruginosa of at least 5%. Treatment of P. aeruginosa overgrowth means that the bacterial load of P. aeruginosa reduces in time upon ingestion of the antibiotic or probiotic composition according to the present invention compared to the bacterial load of P. aeruginosa prior to ingestion of the antibiotic or probiotic composition. Preferably treatment means that the bacterial load of P. aeruginosa reduces to a value below 5%, preferably to a value below 2%, more preferably reduces to the average bacterial load of P. aeruginosa in healthy subjects, in particular healthy subjects who are not diagnosed as having IBS. In practise this means that P. aeruginosa overgrowth is present when P. aeruginosa is detectable in the small intestine or faeces e.g. by the PCR methods as described in example 1 and that treatment means that the bacterial load in faeces and/or mucosa is reduced to practically undetectable levels.

In a preferred embodiment the present invention concerns a method for the treatment of diarrhea, infections, preferably gastrointestinal infections, colics, abdominal cramps and/or abdominal pain in patients with IBS, said method comprising administering a composition comprising one or more probiotic bacteria to a patient need thereof. In other words the invention relates to the use of a composition comprising probiotic bacteria for the preparation of a composition, preferably a nutritional composition, for the treatment of diarrhea, infections, preferably gastrointestinal infections, colics, abdominal cramps and/or abdominal pain in patients with IBS. The invention can also be worded as a composition, preferably a nutritional composition, comprising probiotic bacteria for use in the treatment of diarrhea, infections, preferably gastrointestinal infections, colics, abdominal cramps and/or abdominal pain in patients with IBS.

The inventors also found that the probiotic bacteria are not equally suitable for the treatment of Pseudomonas overgrowth in patients with gut motility disorders such as IBS. It was found, see table 1, that Lactobacillus fermentum NumRes 4, Lactobacillus fermentum NumRes 2, Bifidobacterium animalis BB-12, Bifidobacterium longum biotype longum BB-536 are strong inhibitors of Pseudomonas adhesion to intestinal cells. Therefore, a preferred composition for use in the treatment of Pseudomonas aeruginosa overgrowth in patients with IBS and for the treatment of diarrhea, infections, preferably gastrointestinal infections, colics, abdominal cramps and/or abdominal pain in patients with IBS, comprises Lactobacillus fermentum (NumRes 4), Lactobacillus fermentum (Nu m Res 2), Bifidobacterium animalis (BB-12), Bifidobacterium longum (BB-536) or a mixture thereof. Bifidobacterium animalis (BB-12) can be obtained from Chr. Hansen, Denmark, Bifidobacterium longum (BB-536) is obtained from Morinaga, Japan. In one embodiment the invention concerns the use of a composition comprising Lactobacillus fermentum NumRes 4 (LMG-P-24701), Lactobacillus fermentum NumRes 2 (LMG-P-24701), Bifidobacterium animalis BB-12, or a mixture thereof for the manufacture of a nutritional composition for the treatment of irritable bowel syndrome.

Lactobacillus casei CRL-431 is also a good candidate since this probiotic has been shown to have good adhesive properties on small intestinal cells (Morata et al. (1999) J Food Prot 62: 1430-1434) and has been shown capable to prevent Pseudomonas infections in mice (Alvarez et al. (2001) J Food Prot 64: 1768-1774). Therefore in a preferred embodiment the probiotic composition according to the invention comprises Lactobacillus casei (CRL-431). Lactobacillus casei CRL-431 can be obtained from Chr. Hansen, Denmark. Likewise, due to their beneficial activity, in a preferred embodiment the probiotic composition according to the invention comprises Lactobacillus casei (DN-114 001 =CNCM 1-1518) and/or Bifidobacterium animalis (DN-173 010=CNCM 1-2494). These two have been shown to posses very good probiotic properties such as stability in fresh dairy products and colonization of the intestines.

Lactobacillus fermentum NumRes 4 and Lactobacillus fermentum NumRes 2 have been deposited by the applicant under the Budapest Treaty at the BCCM, (Belgian Coordinated Collections of Microorganisms, Laboratorium voor Microbiologie—Bacterienverzameling (LMG), University of Gent, K. L. Ledeganckstraat 35, B-9000 Gent, Belgium) on 8 Jul. 2008. NumRes 4 has been given BCCM accession number LMG-P-24701. NumRes 2 has been given BCCM accession number LMG-24700. In one embodiment the invention concerns Lactobacillus fermentum NumRes 4 having BCCM accession number LMG-P-24701. In one embodiment the invention concerns Lactobacillus fermentum NumRes 2 having BCCM accession number LMG-P-24700. In one embodiment the invention concerns NumRes 4 (LMG-24701) for use as a medicament. In one embodiment the invention concerns NumRes 2 (LMG-24700) for use as a medicament.

### Nutritional Compositions

In a preferred embodiment, the compositions according to the present invention, and in particular the probiotic compositions, are nutritional compositions. Nutritional compositions preferably comprise at least one, preferably at least two, more preferably at least three macronutrients selected from protein, fat and digestible carbohydrate. In an advantageous embodiment the present nutritional compositions comprise indigestible carbohydrates, also hereinafter referred to as dietary fibers.

Fat

In one embodiment the present nutritional composition comprises fat. Gamma-linoleic acid (GLA) has a stimulating effect on the digestive system operation, as well as the liver and spleen. A preferred embodiment of the present composition therefore comprises a source of GLA. Evening primrose oil is a natural vegetable oil, derived from evening primrose seeds is a suitable source of GLA.

Too much fat in the composition can cause digestive problems that would increase IBS symptoms. Therefore the present composition is preferably low in fat, preferably comprises less than 3 wt % fat, preferably between 0.1 and 3 wt % and even more preferable between 0.1 and 1 wt % fat based on the weight of the total composition.

Protein and Digestible Carbohydrate

In one embodiment the present nutritional composition comprises protein. In one embodiment the present nutritional composition comprises digestible carbohydrate. Advantageously the present composition comprises milk proteins. Many IBS patients are lactose intolerant and these IBS patients would greatly benefit of a diet low in lactose. The present composition therefore preferably comprises a source of milk proteins that is low in lactose or even more preferably is lactose free. Alternatively, a protein source can be used from non-milk origin such as soy. Besides lactose, the present composition preferably comprises digestible carbohydrate selected from the group consisting of sucrose, glucose, fructose, corn syrup solids, starch and maltodextrins.

The present invention advantageously provides a composition wherein the fat provides 0.5 to 15% of the total calories, the protein provides 5 to 50% of the total calories, and the digestible carbohydrate component provides 15 to 90% of the total calories.

Dietary Fibers

In one embodiment the present nutritional composition comprises dietary fibers. Dietary fibers have a beneficial effect in reducing symptoms of IBS. Where the term “dietary fibre” is used in the present description, this is meant to include indigestible oligosaccharide and indigestible polysaccharide, but not mono-saccharide and di-saccharide. Dietary fibres as used in this invention are typically resistant to digestion and absorption in the human small intestine with preferably a complete or partial fermentation in the large intestine. Preferably the present composition comprises at least one dietary fibre selected from the group consisting of galactooligosaccharides (GOS) including trans galactooligosaccharides (TOS), inulin, fructooligosaccharides (FOS) including long chain FOS (IcFOS) and short chain FOS (scFOS) and mixtures thereof, xylooligosaccharides, palatinoseoligosaccharide, soybean oligosaccharide, gentiooligosaccharide, pectin, pectate, alginate, chondroitine, hyaluronic acids, heparine, heparane, sialoglycans, fucoidan, fucooligosaccharides, carrageenan, xanthan gum, cellulose, polydextrose (PDX, a non-digestible carbohydrate that has been synthesized from randomly cross-linked glucose and sorbitol), guar gum, arabinoxylan preferably MGN-3 Rice Bran Arabinoxylan Compound according to U.S. Pat. No. 5,560,914, xyloglycan, callose, lignin and/or degradation products thereof.

In one embodiment the present composition comprises at least two different dietary fibres selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, pectin, pectate, chondroitine, hyaluronic acids, sialoglycans, fucooligosaccharides, xanthan gum, polydextrose (PDX), galactomannans and guar gum, arabinoxylan, preferably MGN-3 Rice Bran Arabinoxylan, xyloglycan, callose, and/or degradation products thereof, wherein at least one is selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, gentiooligosaccharide, xanthan gum, arabinoxylan, polydextrose (PDX), galactomannans, guar gum, and/or degradation products thereof.

In a further embodiment the present composition comprises at least three dietary fibres that are selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, pectin, pectate, chondroitine, hyaluronic acids, sialoglycans, fucooligosaccharides, xanthan gum, polydextrose (PDX), galactomannans and guar gum, arabinoxylan, preferably MGN-3 Rice Bran Arabinoxylan, xyloglycan, callose, and/or degradation products thereof, wherein at least two are selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, gentiooligosaccharide, xanthan gum, arabinoxylan, polydextrose (PDX), galactomannans, guar gum, and/or degradation products thereof and the third is an acid oligosaccharide dietary fibre that is selected from the group consisting of pectin, pectate, chondroitine, hyaluronic acids, sialoglycans and fucooligosaccharides and and/or degradation products thereof.

Surprisingly, certain combinations of fibers show better anti adhesive activity. Combinations of galactooligosaccharides (GOS), including trans galactooligosaccharides (TOS), fructooligosaccharides (FOS), including long chain FOS (IcFOS) defined as consisting of 10 or more monosaccharides and short chain FOS (scFOS) defined as consisting of between 2 and 10 monosaccharides, and several combinations with acidic oligosaccharides such as pectin hydrolysates all give better effects than the individual fibres.

Administration of the present composition therefore can be used to achieve one or more of the following physiological effects: a significant increase of Bifidobacteria and/or lactic acid bacteria; a significant increase in lactic acid and/or a significant increase in total SOFA; a significant increase in relative amount of acetate; a significant decrease in relative amount of butyrate; a significant decrease in the sum of isobutyrate, valerate and isovalerate; a decreased formation of gas; a longer and more even fermentation, including fermentation in the most distal parts of the colon, and a high fermentation in the most proximal part of the colon.

Additionally calcium carbonate can be added to prevent gas formation. Preferred composition therefore comprises dietary fibers in combination with calcium carbonate.

The present dietary fibers can advantageously be used in combination with the probiotics selected from the group consisting of Lactobacillus fermentum (NumRes 4), Lactobacillus fermentum (NumRes 2), Bifidobacterium animalis (BB-12), Lactobacillus casei (CRL-431), Bifidobacterium longum (BB-536), Lactobacillus casei (DN-114 001) and Bifidobacterium animalis (DN-173 010) in a method for the treatment and/or prevention of Pseudomonas aeruginosa overgrowth, diarrhoea, (gastrointestinal) infections, colics, abdominal cramps, abdominal pain, and irritable bowel syndrome.

Fermented Milk Product

In a preferred embodiment the present nutritional composition is in the form of a fermented milk product. In a preferred embodiment the fermented milk product is coagulated milk product that results from fermentation of glucose (derived from the hydrolysis of lactose in milk) to lactate preferably by Lactobacillus bulgaricus and Streptococcus thermophilus preferbly in combination with Bifidobacterium longum (BB-536) and or Bifidobacterium animalis (BB-12) resulting in a fermented milk product, such as yogurt. In addition any of the above mentioned pro- and prebiotics can be added to such fermented milk product for the treatment of Pseudomonas aeruginosa overgrowth and/or infection in patients with IBS.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

Increased P. aeruginosa in intestinal and faecal samples of IBS patients

Methods

Sampling, Preparation and Storage

A sterile cytology brush (Uno-Brush, Prince Medical, Ercuis, France) sheathed in a sterile catheter was placed through the endoscope biopsy channel and advanced under direct vision out beyond the endoscope tip. The duodenal mucosa was brushed three times to obtain mucosa-associated bacteria. Brush samples of each subject were obtained from pars descendens and pars horizontalis of the duodenum. After brushing, the brush was pulled back into the sheath of the catheter, the catheter was removed and the brush was immediately cut off the catheter and placed into a sterile tube in liquid nitrogen and stored at −80° C. until analysis.

Faecal samples were obtained before endoscopy. Faecal samples were stored in the home freezers of the subjects and collected immediately before the endoscopy and stored at −80° C. until analysis. Both faecal and brush samples were stored in dry ice prior to microbiological analysis.

DNA Extraction and PCR Amplification

The frozen faeces were thawed and DNA extraction was performed using the Fast DNA Spin kit, (Qbiogene, Irvine, USA) from 0.5 g faecal material. The frozen brush samples were thawed and suspended in 180 μl ATL buffer and vigorously vortexed to extract the attached bacteria. Subsequently, DNA was isolated from the mucosa-associated bacteria using tissue DNAease kit (Qiagen, Venlo, The Netherlands) according to the manufacture's instructions. The isolated DNA solution of both faeces and brushes were stored at −20° C. The extracted DNA was used as a template to amplify the V6 to V8 regions of 16S rRNA with primers U968-GC-f and U1401-r described in Muyzer G et al., Appl Environ Microbiol 1993; 59(3):695-700 and Muyzer and Smalla, Antonie Van Leeuwenhoek 1998; 73(1):127-141. PCRs were performed using a Taq DNA polymerase kit from Invitrogen (Invitrogen, Paisley,UK). The reaction mixture consisted of 5 μl 10× PCR buffer,50 mM MgCl₂, 50 μM deoxynucleotide triphosphate, 1.25 U of Taq polymerase, 10 pmol of each primer, and 1 μl of appropriately diluted template DNA in a final volume of 50 μl. Samples were amplified in a PTC-200 PCR system (MJ-Research, Waltham, USA) with the following thermocycling program: 94° C. for 5 minutes; 10 cycles of denaturation at 94° C. for 1 minute, annealing temperature of 65-56° C. for 1 minute (reduction of 1° C. for each cycle), extension at 68° C. for 3 minutes; 33 cycles of 94° C. for 1 minute, 56° C. for 1 minute, and 68° C. for 3 minutes. Aliquots of 5 pl PCR product were analyzed by electrophoresis on a 1.5% (wt/vol) agarose gel containing ethidium bromide.

DGGE Analysis of PCR Amplicons

PCR amplicons were separated by DGGE based on the protocol of Muyzer and Smalla (supra) using the DGGE Decode system (Bio-Rad Laboratories, Hercules, Calif., USA) with the following modifications. Polyacrylamide gels consisted of 8% (vol/vol) polyacrylamide (ratio of acrylamide-bisacrylamide; 37.5:1) and 0.5× Tris-acetate-EDTA (pH 8.0) (TAE) buffer. Denaturing acrylamide of 100% was defined as 7 M urea and 40% formamide. The polyacrylamide gels were made with denaturing gradient ranging from 32.5 to 72.5%. The gels were poured from the top using a gradient marker and a pump (Econo gradient pump; Bio-Rad Laboratories, Hercules, Calif., USA) set at a speed of 3 ml/min. Prior to the polymerization of the denaturing gel (gradient volume, 28 ml), a 7.5 ml stacking gel without denaturing chemicals was added, and the appropriate comb was subsequently inserted.

Electrophoresis was performed first for 5 min at 200V and then at 80V for 17 hours in 0.5× TAE buffer at a constant temperature of 60° C. The gels were stained with AgNO₃ and dried overnight at 50° C.

Analysis of the DGGE Profiles and Generation of Pooled Profiles

Standard references were used for normalization of gel profiles. This normalization step enabled a comparison between DGGE profiles from different gels since the DGGE gels were run under comparable denaturing and electrophoretic conditions. Using GelCompar software (Applied Maths, Kortrijk, Belgium), DGGE profiles generated from IBS patients and healthy subjects were pooled together, in an average IBS fingerprint and an average healthy subject fingerprint respectively, based on band intensity and position in the gel. Band differences in these average profiles were determined and the bands which were not identical in the average IBS and healthy profile were localized in the individual DGGE profiles. The most intense bands in the individual profiles were cut from the original gels. The DNA of these bands was used for a second DGGE analysis using a 46-51.6% gradient. Bands of in terest were subjected to sequencing as described below.

Cloning and Sequencing DNA from DGGE Bands

In order to identify the bacteria correlated to the bands, a small piece from the middle of the selected band was cut from the DGGE gel with a sterile scalpel and then incubated in 50 μl sterile Milli-Q for 24 hours at 4° C. to allow diffusion. The eluent containing DNA fragments was used for PCR reamplification with the same primers used earlier. To check whether the DNA of interest on the first DGGE gel and the reamplified DNA migrated to the same position, a second DGGE was carried out in order to compare the two samples. When the two bands comigrated, DNA fragments were purified with the GenElute PCR DNA Purification Kit (Sigma, Zwijndrecht, The Netherlands) and thereafter ligated into the pCR® 2.1-TOPO®vector and transformed into E. coli One Shot TOP10 competent cells (Invitrogen, Paisley, UK). Plasmids from colonies of kanamycin resistant transformants were extracted with the Qiagen Plasmid Midi Purification Kit (Qiagen, Venlo, The Netherlands). The extracted plasmids were screened for inserts of the correct size by performing a PCR with the M13 forward and reverse primers as well as with the 968f and 1401r primers. Insert PCR amplicons of selected transformants were purified and were subjected to DNA sequence analysis. (Baseclear, Leiden, The Netherlands). Sequence similarities of the clones were checked with the Basic Local Alignment Search Tool (BLAST) at the NCB! database http://www.ncbi.nlm.nih.gov/BLAST).

Real Time q-PCR Analysis

Quantitative Real Time PCR (q-PCR) was performed to determine the percentages of P. aeruginosa in brush and faecal samples. Measurement of P. aeruginosa was performed as described by Pirnay et al., Crit Care 2000; 4(4):255-261, with some slight modifications. The fluorescent labels were changed from LC Red 640 to 6FAM and from fluorescein to TAMRA while the other ingredients and conditions for the described q-PCR stayed the same. The fluorescence signal was measured in the annealing phase on the ABI 7900HT Fast (Applied Biosystems, Nieuwerkerk aan de Ussel, The Netherlands). The total bacterial load was determined as described by Nadkarni et al. Microbiology 2002; 148(Pt 1):257-266. The relative percentage of P. aeruginosa was subsequently calculated according to Liu et al. Biochem Biophys Res Commun 2002; 294(2):347-353 and Anal Biochem 2002; 302(1):52-59. The efficiency of each amplification curve was calculated separately and used to determine the initial amount of DNA. Finally, the obtained ratios between the initial amounts of DNA were normalized against a monoculture of the same species, which was set at 100%.

Statistical Analysis

DGGE data were analyzed by GelCompar II software (Applied Maths, Belgium). Cluster analysis and calculation of the similarity indices between the different banding patterns were performed using Pearson product-moment correlation and the unweighted-pair group method using arithmetic averages (UPGMA).

Pseudomonas aeruginosa levels of IBS patients and healthy subjects were analyzed using independent samples t-test. Pseudomonas aeruginosa levels of IBS subgroups were analyzed using one-way ANOVA with Bonferroni correction. Data are expressed as mean ±SEM. SPSS 12.0.1 for Windows was used for analysis

Results

DGGE gels were identical for 78.2% in the small intestinal samples and for 86.25% in the faecal samples of both groups. Clones of small intestinal and faecal bands confined to IBS were mainly identified as Pseudomonas species of which Pseudomonas aeruginosa was the predominant species.

Percentages P. aeruginosa of total bacterial load in the small intestine of IBS patients were significantly higher (8.3±0.950%) than in Healthy Subjects (0.1±0.069%) (P<0.001). In faeces of IBS patients percentages P. aeruginosa of total bacterial load were also significantly higher (2.34±0.31%) than in Healthy Subjects (0.003±0.0027%) (P<0.001).

### Conclusion

The results show that P. aeruginosa is increased in mucosa-associated small intestinal and faecal samples of IBS patients, clearly indicating a role of P. aeruginosa in the pathophysiology of IBS.

### Example 2

Selecting of probiotic strains for optimal inhibition of Pseudomonas aeruginosa adhesion to human intestinal cells (Caco-2)

Method

Caco-2 cells were grown in a 24-wells plate in MEM+ medium with 20% FCS (fetal calf serum) at 37° C. in a CO₂ incubator for 2 weeks until a monolayer was established. MEM+medium contains MEM (Gibco 10938) supplemented with 10 mM Na-pyruvate, 1× non-essential amino acids (Gibco 11130-36) and 100 U penicillin—100 μg streptomycin (penstrep; Gibco 15140-130). One day before the experiment the medium was replaced with MEM+ medium (penstrep free) containing 10% FCS and 1 h before the experiment the medium was replaced with MEM+ medium (penstrep free) containing 1% FCS. Overnight cultures of Pseudomonas aeruginosa P112 in TSB broth and lactic acid bacteria in rMRS broth were washed once in PBS, counted microscopically and diluted to stocks of 2×10E8 CFU/ml and 1×10E9 CFU/ml respectively in the Caco-2 cell culture. The P112 stock culture was also plated in appropriate dilutions on NA-agar. 250 μl of Caco-2 stock culture was mixed with 250 μl of lactic acid bacterial stock. Thereafter 250 μl of the P112 culture was added and the mix was incubated for 1 h at 37° C. in a CO₂ incubator. As a negative control Caco-2 cells were incubated with P112 w/o addition of lactic acid bacteria. After incubation the cells were washed three times in PBS to remove no-adherent bacteria. Distilled water was added to detach the bacteria and lyse the Caco-2 cells. Samples were subsequently plated in appropriate dilutions on NA-agar to determine the P112 viable counts and to calculate the percentage of adhered bacteria.

Results

The influence of probiotic strains on the adherence of Pseudomonas aeruginosa to intestinal epithelial cells was determined by in-vitro experiments using Caco-2 cells. In these experiments P. aeruginosa strain P112 was used which is a clinical isolate (AZU R872). Caco-2 cells are human colonic adenocarcinoma cells that are able to express differentiation features characteristic of mature intestinal cells and therefore valuable in-vitro tools for studies related to intestinal cell function. Co-incubation of Caco-2 cells with P112 and B. longum BB-536 showed an inhibition of 61% of Pseudomonas adherance to the cells. Other strains of lactic acid bacteria were also inhibiting Pseudomonas adherence but to a lesser extent. The results are also summarised in Table 1. These results show that B. longum BB-536 is capable of suppressing colonization of P. aeruginosa in the upper intestine.

TABLE 1
Inhibition (%) of P. aeruginosa P112 adherence
to Caco-2 cell monolayers co-incubated with various
strains of lactic acid bacteria.

| | | | Inhibition of | |
|---|---|---|---|---|
| NumRes | | | adhesion of | Avail- |
| code | Source | Species | P. aeruginosa (%) | ability |
| | BB-536 | Bifidobacterium | 61 ± 3 (n = 3) | Morinaga |
| | | longum | | Milk |
| | | | | Industry |
| NumRes | Danone | Bifidobacterium | 31 ± 6 (n = 2) | Danone |
| 276 | isolate | bifidum | | |
| | BB-12 | Bifidobacterium | 42 ± 12 (n = 3) | Chr. |
| | | animalis | | Hansen |
| NumRes | LW744 | Lactobacillus | 38 ± 21 (n = 6) | Danone |
| 1 | (Lab | rhamnosus | | |
| | Wisby/) | | | |
| NumRes | Danone | Lactobacillus | 45 ± 22 (n = 4) | BCCM |
| 2 | isolate | fermentum | | |
| NumRes | Danone | Lactobacillus | 52 ± 8 (n = 4) | BCCM |
| 4 | isolate | fermentum | | |
| NumRes | Danone | Lactobacillus | 38 ± 5 (n = 3) | Danone |
| 6 | isolate | rhamnosus | | |
| NumRes | Danone | Pediococcus | 29 ± 5 (n = 2) | Danone |
| 3 | isolate | acidilactici | | |

Fermentation pattern used to characterize the different strains of Lactobacillus fermentum (NumRes 2 and NumRes 4) indicating that the isolates are different:

| | NumRes 2 | NumRes 4 |
|---|---|---|
| Ribose | + | + |
| Galactose | − | + |
| Glucose | + | + |
| Fructose | + | − |
| Mannose | + | + |
| Maltose | + | − |
| Lactose | + | + |
| Melibiose | − | − |
| Sucrose | + | + |
| Trehalose | − | − |
| L-arabinose | − | − |
| Mannitol | − | − |

Furthermore it is known that oral administration of B. longum protects mice against gut derived sepsis caused by P. aeruginosa, and the effect may be due to interference of P. aeruginosa adherence to intestinal epithelial cells, see Matsumoto et al. (2008) J. Applied Microbiology 104: 672-680. This shows the capability of this specific probiotic bacterium to decrease P. aeruginosa in the gut and thus its suitability for treating IBS.

It is known that Lactobacillus casei CRL-431 has good adhesive properties on small intestinal cells and is capable to prevent Pseudomonas infections in mice. This shows the capability of this specific probiotic bacterium to decrease P. aeruginosa in the gut and thus its suitability for treating IBS.

Additionally, anti-pseudomonas actitvity has been found for Lactobacillus casei (DN-114 001=CNCM I-1518) and Bifidobacterium animalis (DN-173 010). Consequentlly, this shows the capability of these specific probiotic bacteria to decrease P. aeruginosa in the gut and thus their suitability for treating IBS as well.

1-16. (canceled)

17. A method for the treatment of Pseudomonas aeruginosa overgrowth in a patient with a gut motility disorder comprising administering to the patient a composition comprising probiotic bacteria selected from the group consisting of Lactobacillus fermentum NumRes 4, Lactobacillus fermentum NumRes 2, Lactobacillus casei CRL-431, Bifidobacterium animalis BB-12, Bifidobacterium longum BB-536, Lactobacillus casei DN-114 001, Bifidobacterium animalis DN-173 010 and mixtures thereof.

18. The method according to claim 17, wherein the gut motility disorder is irritable bowel syndrome.

19. The method according to claim 18, wherein the patient exhibits diarrhea, infections, colics, abdominal cramps and/or abdominal pain.

20. The method according to claim 17, wherein the probiotic bacteria is selected from the group consisting of Lactobacillus fermentum NumRes 4 (LMG-P-24701), Lactobacillus fermentum NumRes 2 (LMG-P-24700), Bifidobacterium animalis BB-12, and mixtures thereof.

21. The method according to claim 17, wherein the composition further comprises dietary fibres.

22. The method according to claim 21, wherein the dietary fibres are selected from the group consisting of galactooligosaccharides (COS), trans-galactooligosaccharides (TOS), fructooligosaccharides (FOS) and pectin hydrolysate.

23. The method according to claim 17, wherein the composition further comprises protein, digestible carbohydrate and/or fat.

24. The method according to claim 17, wherein the composition further comprises between 0.1 and 3 wt % fat, based on the weight of the composition.

25. The method according to claim 17, wherein the composition is a fermented milk product.

26. The method according to claim 17, wherein the Lactobacillus fermentum NumRes 4 comprises Lactobacillus fermentum having BCCM accession number LMG-P-24701.

27. The method according to claim 17, wherein the Lactobacillus fermentum NumRes 2 comprises Lactobacillus fermentum having BCCM accession number LMG-P-24700.

28. A method for the treatment of irritable bowel syndrome, comprising administering to a patient in need thereof a composition comprising of Lactobacillus fermentum NumRes 4, Lactobacillus fermentum NumRes 2, Bifidobacterium animalis BB-12, or a mixture thereof.

29. A method for the treatment of irritable bowel syndrome, comprising administering to a patient in need thereof a composition comprising one or more anti-Pseudomonas antibiotics.

30. The method according to claim 29, wherein the one or more antibiotics is a combination of an anti-Pseudomonas beta-lactam and an aminoglycoside or fluoroquinolone.

31. The method according to claim 29, wherein the one or more antibiotics is selected from the group consisting of carbapenem, aminoglycoside, Cotrimox®, Ciprofloxacin and Norfloxacin.
